(19) United States
(12) Patent Application Publication
(10) Pub. No.: US 2025/0057625 A1
(43) Pub. Date: Feb. 20, 2025

(54) SEPARATION DEVICE, SYSTEMS, AND METHODS

(71) Applicants: Sarah Jane Ellsworth, Aurora, CO (US); Samuel John Ellsworth, Boulder, CO (US)

(72) Inventors: Sarah Jane Ellsworth, Aurora, CO (US); Samuel John Ellsworth, Boulder, CO (US)

(21) Appl. No.: 18/806,576

(22) Filed: Aug. 15, 2024

Related U.S. Application Data
(60) Provisional application No. 63/520,225, filed on Aug. 17, 2023.

(51) Int. Cl. A61B 90/00

(52) U.S. Cl. CPC A61B 90/08

(57) ABSTRACT

The present disclosure relates generally to a separation device, systems, and methods of manufacturing and use thereof. In some embodiments, the separation device includes: a spreader assembly including two arms, an arm connector (e.g., a pin), and attaching means, and two pads (or cups) to attach to the spreader assembly. In some embodiments, the separation device may be used to separate a patient's buttocks for examination or procedural purposes.

### CROSS-REFERENCE TO RELATED APPLICATION

This application claims the benefit of U.S. Provisional Patent Application No. 63/520,225, entitled “SEPARATION DEVICE, SYSTEMS, AND METHODS,” filed on 17 Aug. 2023, which is specifically incorporated by reference for all it discloses and teaches.

### FIELD OF TECHNOLOGY

The present disclosure relates generally to a separation device, systems, and methods of manufacturing and use thereof.

### BACKGROUND

During anal region and rectal medical exams or procedures, a treating provider is required to separate the buttocks for access to the anal region. The treating provider separates the buttocks with their hands. This separation can cause discomfort for the patient, and it can be difficult for the treating provider to create and maintain a desired separation while accessing the anal region. Other methods to separate the buttocks are desirable.

### SUMMARY

The present disclosure relates generally to a separation device, systems, and methods of manufacturing and use thereof. In some embodiments, the disclosure is directed to a separation device that separates the buttocks with the use of pads (or cups) attached to a spreader assembly.

In some embodiments, the separation device includes a spreader assembly, including: two arms, a pin connector connecting both of the arms to each other and allowing for rotation of each arm about the connecting pin. The separation device may include inger loops located on the two arms. A cup may be attached to an end of each arm. In some embodiments, the cups may include at least one of metal, highly resilient foam, elastomers, gel, air, firm memory foam, silicone, or other appropriate material for contacting the skin.

In some embodiments, the cups are configured to freely rotate on the spreader assembly.

In some embodiments, the arms of the spreader assembly are adjustable to a variety of distances between the cups.

In some embodiments, the cups are detachable from the spreader assembly.

In some embodiments, the cups are interchangeable and varying in sizes.

In some embodiments, the spreader assembly is configured to receive various sized cups.

In some embodiments, the spreader assembly includes a locking mechanism. The locking mechanism may be a ratcheting system or other type of locking system.

In some embodiments, the spreader assembly includes an attaching means to attach the pads to the spreader assembly.

In some embodiments, a method of using a separation device is disclosed. The method may include placing two pads on buttocks of a patient, wherein each pad is placed on each buttock, and operating a spreader assembly on the separation device to separate the buttocks from each other; and positioning the buttocks to desired locations.

The method may include operating the spreader assembly by moving two arms of the spreader assembly attached to the two pads to separate the buttocks and positioning the buttocks to desired locations.

The method may include moving the two arms means utilizing finger loops on the arms to move the arms to place the two pads in a desired location.

The method may include replacing the two pads with new pads.

The method may include locking the spreader assembly in a fixed position with a locking mechanism. The locking mechanism may be a ratcheting system.

In some embodiments, a method of manufacturing a separation device is disclosed. The method may include forming a spreader assembly, wherein forming the spreader assembly includes providing two arms; connecting the arms to each other via an arm connecting pin; forming an attaching means on each of the two arms, while still allowing rotation about the connection; and attaching pads on the attaching means.

The method may include forming handles on the two arms. The handles may be finger loops.

The method may include forming a locking mechanism on the spreader assembly.

These and various other features and advantages will be apparent from a reading of the following detailed description.

### BRIEF DESCRIPTION OF THE DRAWINGS

FIG. 1 is a top view of a separation device in one embodiment according to the present disclosure in an unlocked position.

FIG. 2 is a perspective view of a separation device in one embodiment according to the present disclosure in an unlocked position.

FIG. 3 is a perspective view of a separation device in one embodiment according to the present disclosure in a locked position.

FIG. 4 is a top view of a separation device in one embodiment according to the present disclosure in a locked position.

FIG. 5 is a front view of a separation device in one embodiment according to the present disclosure in a locked position.

FIG. 6 is a perspective view of a separation device in one embodiment according to the present disclosure in an unlocked position.

FIG. 7 is a flowchart of a method of operation of a separation device in one embodiment according to the present disclosure.

FIG. 8 is a flowchart of a method of manufacture of a separation device in one embodiment according to the present disclosure.

### DETAILED DESCRIPTION

In the following description, for the purposes of explanation, numerous specific details are set forth in order to provide a thorough understanding of the present invention. It will be apparent, however, to one skilled in the art that the present invention may be practiced without some of these specific details. For example, while various features are ascribed to particular implementations, it should be appreciated that the features described with respect to one implementation may be incorporated with some implementations as well. Similarly, however, no single feature or features of any described implementation should be considered essential to the invention, as some implementations of the invention may omit such features.

As used herein, the singular forms “a,” “an.” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “lower,” “upper,” “beneath,” “below,” “above,” “on top,” “front,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

The present disclosure is directed to a separation device that separates the buttocks with the use of pads (or cups) attached to a spreader assembly.

In some embodiments, the separation device includes: a spreader assembly including two arms, an arm connector (e.g., a pin), and attaching means, and two pads (or cups) to attach to the spreader assembly.

In some embodiments, the pads (or cups) attach to the spreader assembly by clasps, clips, snaps, hooks, or other attaching means. The pads may be freely rotating on the attaching means to accommodate a desired separation or anatomy, or fixed. In some embodiments, the pads (or cups) may be made out of metal, plastic, or other materials.

It should be appreciated that the pads (or cups) can be any mechanism to hold the buttocks apart. The pads can be of various shapes and materials. For example, the pads may be shaped to align where the pad will contact the buttocks. For another example, the pads may be loops. The pads may also be of various thicknesses. The pads may also include metal, highly resilient foam, elastomers, gel, air, firm memory foam, silicone, or a combination.

In some embodiments, the device includes no cups on the end of the arms and another mechanism, such as metal loops, hold the buttocks apart.

In some embodiments, the device includes reverse ratcheting bar clamp or a c-clamp.

In some embodiments, the pads include a sleeve, cover, or coating to increase patient comfort and prevent slippage, or for reuse or skin protection. In some embodiments, the pads can be covered with a disposable, washable, or otherwise replaceable covering for hygienic and sterile use with new patients. In some embodiments, a disposable sheet of paper may be fit over the pads for each use.

In some embodiments, the separation device includes a spreader assembly to attach to the pads, and move the pads in a desired placement. The spreader assembly may be any device capable of holding the pads against the buttocks and enabling separation. In some embodiments, the spreader assembly can be a reverse ratcheting bar clamp, or a c-clamp, for example.

In some embodiments, the spreader assembly has two arms, and each arm connects to a pad. The arms may be moved to control the placements of the pads. The arms may be attached to one another via an arm connector. The arm connector may be located in the center of the arms or any other location on the length of the arm that permits desired placement of the pads on the buttocks and allows for arm rotation about the connection.

In some embodiments, the two arms of the spreader assembly each have a finger loop on one end of each arm for a user to grasp with fingers to operate the spreader assembly. In other embodiments, the user may operate the arms via handles or other grasping mechanism. A user may use the finger loops, handles, or other grasping mechanism to move the arms in different positions.

The arm connector may be a rotational pin to facilitate rotation of the arms with respect to each other. In some embodiments, the arm connector is located medially in both arms. In some embodiments, the arm connector may separate a top portion from a bottom portion of each arm.

In some embodiments, the arms are pivotably connected to each other about a hinge pin. The hinge pin includes a first end, a second end, and a central longitudinal axis extending therebetween allowing for rotation.

In some embodiments, the top portion of each arm may be a substantially straight, and the lower portion may be substantially curved. The finger loops may be located proximate to the top portion of the arms. The lower curved portion ends may have attachments to attach the cups to the spreader assembly. In some embodiments, the entire arm may be a substantially straight.

In some embodiments, the two arms have a locking mechanism located in the top, middle, or lower portions of the arms that locks the arms to hold the buttocks apart once separated so that the separation device (and buttocks) will remain in a fixed position. In some embodiments, the locking mechanism may be a ratcheting system. For example, the ratcheting system may be ratchet finger ring handles. In another example, the ratcheting system may be located on the arm below the finger loops, and a component of the ratchet on each finger loop may attach to the other component for clamping. It should be appreciated that any suitable locking mechanism may be used to hold the separation device (and buttock) in a fixed position, such as a latch and pin system, for example.

When the user has two fingers of the same hand in the finger loops, they can approximate those fingers. In some embodiments, when the user approximates their fingers in the finger loops on one end of the arms, bringing the fingers loops toward each other in a closed configuration, the pads on the other end of the arms are spread apart from each other. This action spreads the pads apart, which separates the buttocks.

In some embodiments, the pads may separate when the fingers loops separate. As the user moves the finger loops away from each other, the pads move away from each other.

The user can use the locking mechanism to lock the device in this conformation. The locking mechanism can be easily unlocked to reverse this action. In some embodiments, the approximation of the fingers on the proximal end of the spreader allows rotation about a pin in the middle of the device (midshaft along the spreader arms), which allows angle widening of the distal arms (with the pads attached).

In some embodiments, the pads may be spread apart from one another in varying distances and angles. For example, the arms at the end of the spreader assembly may be configured with the cups attached and angled outwards (away from one another), curving downward (towards/perpendicular to the patient) to enable the spreading capability of the device and ensure that the user interface of the device is out of the way and angled parallel to the patient rather than perpendicular to keep the field of vision around the anus of the treating provider clear for procedural or examination purposes.

In some embodiments, the materials in the separation device may be autoclaved to ensure sterility and reusability.

In some embodiments, the two arms are mirror images of each other.

In some embodiments, the shape of the cups is such that the lowest portion of the cups (located closest to a patient's anus during use) is angled more medially than the top portion of the cups (located more caudally on the patient during use).

In some embodiments, each cup is concave on the portion that abuts the buttock to naturally mimic the anatomical curve of the buttock.

In some embodiments, the spreader assembly may only have one arm that has two attachment areas for pads. The user may position the spreader assembly against the buttocks and separate the buttocks merely by pressing the pads against the buttocks.

### METHODS OF USE

In one embodiment, to operate the separation device, a user inserts fingers (e.g., a finger and a thumb) into the finger loops on the proximal end of arms. The movement of opening and closing the finger and thumb with respect to each other actuates the movement of the device. When a user opens and closes the fingers, the distance between the distal ends of the spreader (where the pads are attached) changes. Increased finger approximation results in increased distance between the spreading cups. Similarly, decreased finger approximation results in decreased distance between the spreading cups. In some embodiments, increased finger approximation results in decreased distance between the spreading cups, and decreased finger approximation results in increased distance between the spreading cups.

When the finger and thumb are approximated and the buttocks are separated, the locking mechanism can maintain the spreaders in a desired orientation. The locking mechanism is of a sufficient length that various angles between the distal arms of the separation device can be achieved to adjust for varying anatomic buttocks sizes and separability. In some embodiments, any desired angles and positioning can be changed during use. In some embodiments, there are predetermined angles and positioning incorporated into the device. For example, there may be setting for a limited number of angles and positions. In some embodiments, the locking mechanism can be adjusted such that when the pads are further apart, the more the locking mechanism clicks together. In some embodiments, the locking mechanism can be unlocked by a user flexing the finger loops away from one another in the direction perpendicular to the orientation of the device arms.

### Methods of Manufacturing

A method of manufacturing a separation device may include forming a spreader assembly, wherein forming the spreader assembly includes providing two arms, connecting the arms to each other via an arm connector, forming an attaching means on each of the two arms, and attaching pads on the attaching means. The method may include forming handles on the two arms. The handles may be finger loops. The method may include forming a locking mechanism on the spreader assembly.

These and various other features and advantages will be apparent from a reading of the following detailed description.

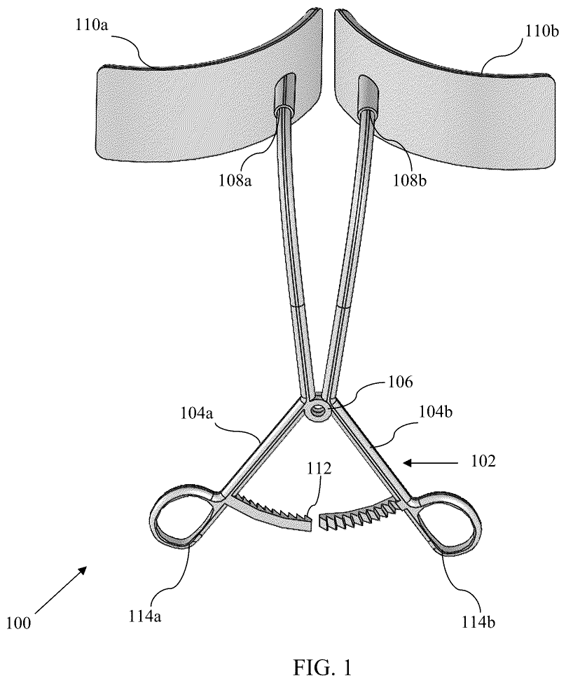

Referring to FIG. 1, a top view of a separation device 100 in one embodiment according to the present disclosure in an unlocked position is shown. The separation device 100 includes a spreader assembly 102 which includes two arms 104a and 104b, an arm connector (e.g., a pin) 106, attaching means 108a and 108b, and two pads (or cups) 110a and 110b to attach to the spreader assembly. The spreader assembly 102 includes a locking mechanism 112. The spreader assembly 102 includes handles 114a and 114b.

In some embodiments, the arms 104a and 104b may be curved for user fit and comfort. The arm connector 106 may include a rotation pin, or any other means for pivotably, rotatably, or otherwise separably connecting the arms 104a and 104b. The attaching means 108a and 108b may include freely rotating joints. The two pads (or cups) 110a and 110b may be any mechanism that can be used to spread buttocks apart when contact is made, such as loops. The two pads (or cups) 110a and 110b may be freely detachable, locked into place, or permanently installed. In some embodiments, the handles 114a and 114b may be finger loops.

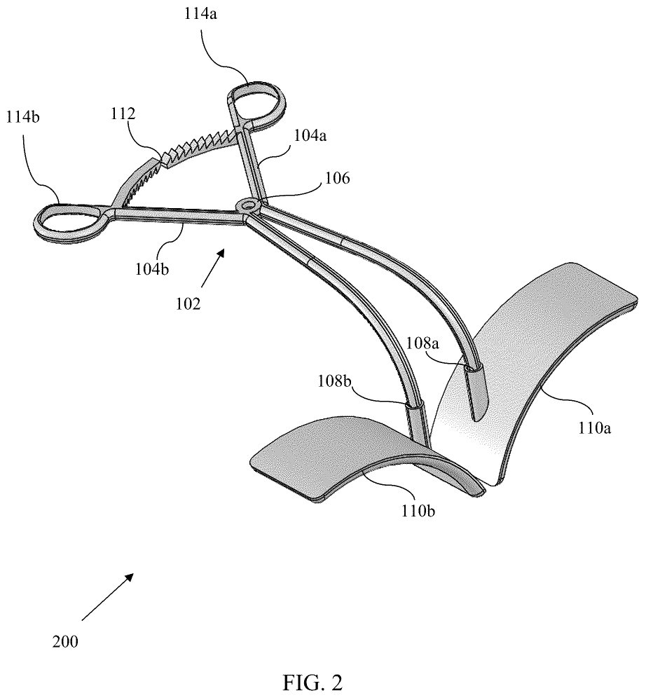

Referring to FIG. 2, a perspective view of a separation device 200 in one embodiment according to the present disclosure in an unlocked position is shown. The separation device 200 includes a spreader assembly 102 which includes two arms 104a and 104b, an arm connector (e.g., a pin) 106, attaching means 108a and 108b, and two pads (or cups) 110a and 110b to attach to the spreader assembly. The spreader assembly 102 includes a locking mechanism 112. The spreader assembly 102 includes handles 114a and 114b.

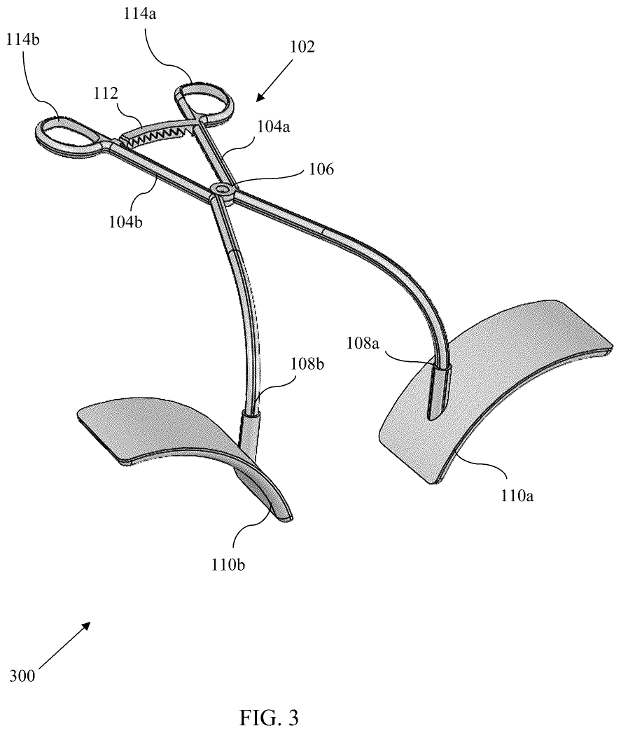

Referring to FIG. 3, a perspective view of a separation device 300 in one embodiment according to the present disclosure in a locked position is shown. The separation device 300 includes a spreader assembly 102 which includes two arms 104a and 104b, an arm connector (e.g., a pin) 106, attaching means 108a and 108b, and two pads (or cups) 110a and 110b to attach to the spreader assembly. The spreader assembly 102 includes a locking mechanism 112. The spreader assembly 102 includes handles 114a and 114b.

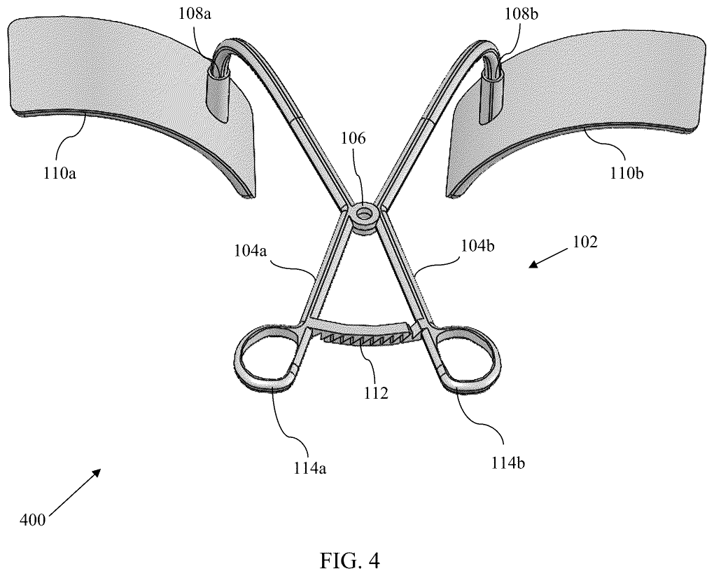

Referring to FIG. 4, a top view of a separation device 400 in one embodiment according to the present disclosure in a locked position is shown. The separation device 400 includes a spreader assembly 102 which includes two arms 104a and 104b, an arm connector (e.g., a pin) 106, attaching means 108a and 108b, and two pads (or cups) 110a and 110b to attach to the spreader assembly. The spreader assembly 102 includes a locking mechanism 112. The spreader assembly 102 includes handles 114a and 114b.

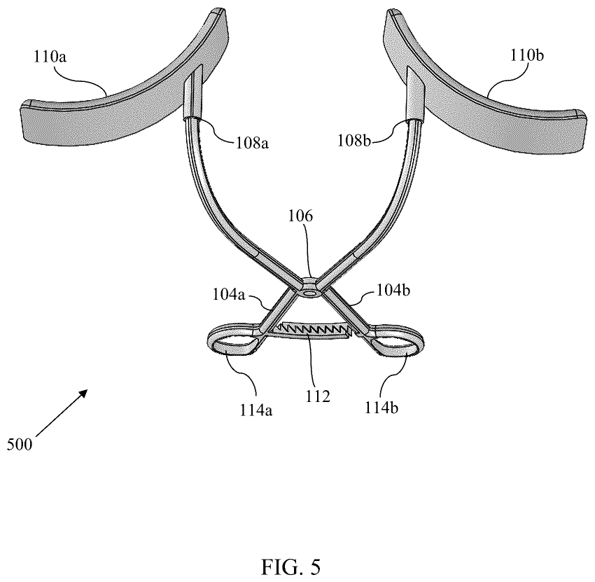

Referring to FIG. 5, a front view of a separation device 500 in one embodiment according to the present disclosure in a locked position is shown. The separation device 500 includes a spreader assembly 102 which includes two arms 104a and 104b, an arm connector (e.g., a pin) 106, attaching means 108a and 108b, and two pads (or cups) 110a and 110b to attach to the spreader assembly. The spreader assembly 102 may include a locking mechanism 112. The spreader assembly 102 may include handles 114a and 114b.

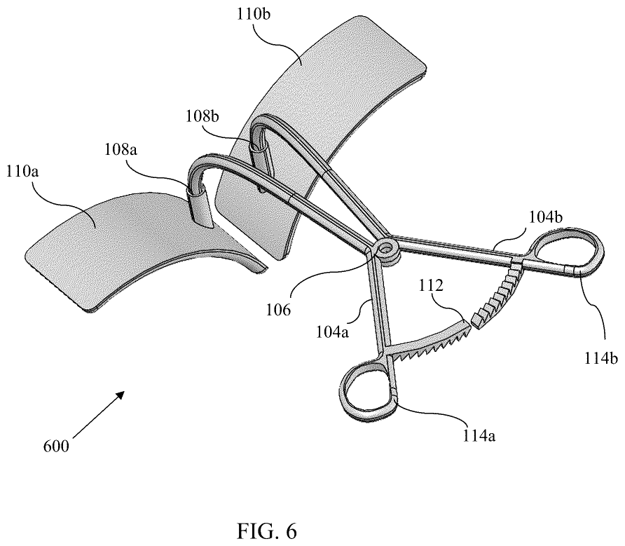

Referring to FIG. 6, a perspective view of a separation device 600 in one embodiment according to the present disclosure in an unlocked position is shown. The separation device 600 includes a spreader assembly 102 which includes two arms 104a and 104b, an arm connector (e.g., a pin) 106, attaching means 108a and 108b, and two pads (or cups) 110a and 110b to attach to the spreader assembly. The spreader assembly 102 includes a locking mechanism 112. The spreader assembly 102 includes handles 114a and 114b.

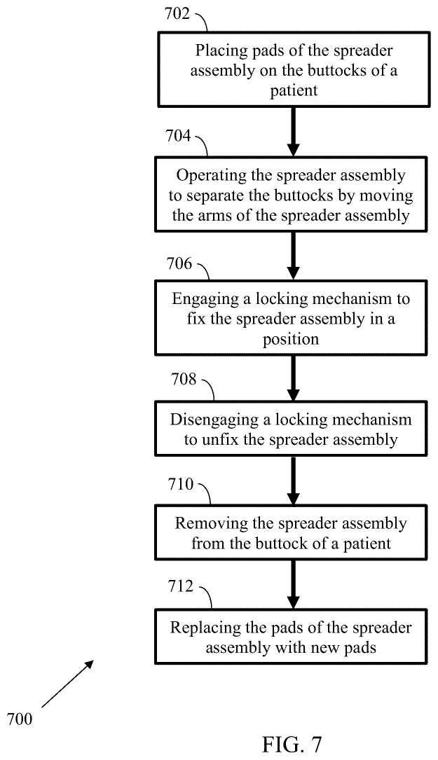

Referring to FIG. 7, a flowchart of a method of operation 700 of a separation device in one embodiment according to the present disclosure is shown. At 702, pads of a spreader assembly may be placed on the buttocks of a patient. In some embodiments, the pads of the spreader assembly may be adjusted to best fit the buttocks of the patient. In some embodiments, the pads may be adjusted to predetermined positions and angles.

At 704, an operator may operate the spreader assembly to change a distance between the buttocks of the patient by actuating or otherwise moving the arms of the spreader assembly. In some embodiments, decreasing a distance between handles of a spreader assembly can cause the pads of the spreader assembly to separate and position the buttocks to desired locations. In other embodiments, increasing a distance between the handles of the spreader assembly can cause the pads of the spreader assembly to separate. As a result, the separation device can separate the buttocks from each other, and position the buttocks to desired locations.

In some embodiments, the spreader may be fixed. At 706, an operator may engage a locking mechanism to fix the spreader assembly in a desired position or orientation, and accordingly, the pads of the fixed spreader assembly can hold the buttocks of the patient in the desired position or orientation. At 708, the operator may disengage a locking mechanism to unfix the spreader assembly. In some embodiments, increasing a distance between the handles of a spreader assembly can cause the pads of the spreader assembly to approximate or come closer together. In other embodiments, decreasing a distance between the handles of a spreader assembly can cause the pads of the spreader assembly to approximate or come closer together. At 710, the spreader assembly may be removed from the buttocks of a patient. At 712, the pads of the spreader assembly may be replaced with new pads to accommodate sterile or otherwise sanitary use with a new patient. In some embodiments, instead of replacing the pads of the spreader assembly with new pads, the pads of the spreader assembly may be covered with a disposable sheet for each use (e.g., a sheet of paper that is replaced upon each use of the spreader assembly). In some embodiments, the pads of the spreader assembly may not be replaced.

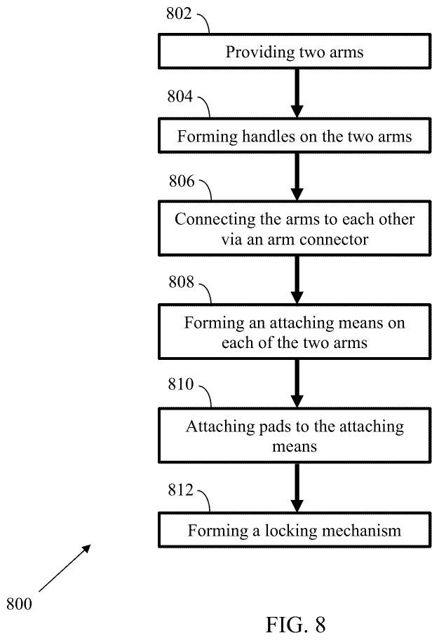

Referring to FIG. 8, a flowchart of a method of manufacture 800 of a separation device in one embodiment according to the present disclosure is shown. At 802, two arms of the spreader assembly may be provided. At 804, handles may be formed on the two arms. In some embodiments, the handles may be finger loops. At 806, the arms may be connected to each other via an arm connector. In some embodiments, the arm connector includes a pin in which the arms may pivot about. At 808, an attaching means may be formed on each of the two arms. In some embodiments, the attaching means may be freely rotatable or otherwise adjustable to accommodate for adjustment of pads. In some embodiments, the attaching means may be fixed. At 810, pads may be attached to the attaching means. In some embodiments, the pads are removably attached to the attaching means, such that new pads may be attached to the attaching means. At 812, a locking mechanism may be formed on the separation device. Any locking mechanism may be formed to fix the separation device in a desired position or orientation. In some embodiments, the locking mechanism may be formed as a ratcheting system.

It should be noted that these methods describe examples of implementations, and that the operations and the steps may be rearranged, omitted, or otherwise modified such that other implementations are possible. In some examples, aspects from two or more of the methods may be combined. For example, aspects of each of the methods may include steps or aspects of the other methods, or other steps or techniques described herein. Thus, aspects of the disclosure may provide for consumer preference and different products.

The description set forth herein, in connection with the appended drawings, describes example configurations and does not represent all the examples that may be implemented or that are within the scope of the claims. The term “example” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and devices are shown in block diagram form in order to avoid obscuring the concepts of the described examples.

The description herein is provided to enable a person having ordinary skill in the art to make or use the disclosure. Various modifications to the disclosure will be apparent to a person having ordinary skill in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

What is claimed is:

1. A separation device to separate the buttocks comprising:
a spreader assembly, including:
two arms;
an arm connector connecting the arms to each other;
finger loops located on the two arms; and
two cups, each cup attached to an end of each arm.

2. The separation device of claim 1, wherein the cups may include at least one of metal, highly resilient foam, elastomers, gel, air, firm memory foam, and silicone.

3. The separation device of claim 1, wherein the cups are configured to freely rotate on the spreader assembly.

4. The separation device of claim 1, wherein the arms of the spreader assembly are adjustable to a variety of distances between the cups.

5. The separation device of claim 1, wherein the cups are detachable from the spreader assembly.

6. The separation device of claim 1, wherein the cups are interchangeable varying sizes.

7. The separation device of claim 1, wherein the spreader assembly is configured to receive various sized cups.

8. The separation device of claim 1, wherein the spreader assembly includes a locking mechanism.

9. The separation device of claim 8, wherein the locking mechanism is a ratcheting system.

10. The separation device of claim 1, further comprising an attaching means to attach the pads to the spreader assembly.

11. A method of using a separation device comprising:
placing two pads on buttocks of a patient, wherein each pad is placed on each buttock;
operating a spreader assembly on the separation device to separate the buttocks from each other; and
positioning the buttocks to desired locations.

12. The method of claim 11, wherein operating the spreader assembly includes:
moving two arms of the spreader assembly attached to the two pads to separate the buttocks.

13. The method of claim 12, wherein moving the two arms means utilizing finger loops on the arms to move the arms to place the two pads in a desired location.

14. The method of claim 11, further comprising:
replacing the two pads with new pads.

15. The method of claim 11, further comprising locking the spreader assembly in a fixed position with a locking mechanism.

16. The method of claim 15, wherein the locking mechanism is a ratcheting system.

17. A method of manufacturing a separation device comprising:
forming a spreader assembly, wherein forming the spreader assembly includes:
providing two arms; and
connecting the arms to each other via an arm connector; and
forming an attaching means on each of the two arms; and
attaching pads on the attaching means.

18. The method of claim 17, further comprising:
forming handles on the two arms.

19. The method of claim 18, wherein the handles are finger loops.

20. The method of claim 17, further comprising:
forming a locking mechanism on the spreader assembly.
